(19) United States
(12) Patent Application Publication
(10) Pub. No.: US 2025/0162970 A1
(43) Pub. Date: May 22, 2025

(54) SEPARATION OF ACRYLIC ACID WITH THE AID OF MEMBRANE CONTACTORS

(71) Applicant: Evonik Superabsorber GmbH, Essen (DE)

(72) Inventors: Jessica Liestener, Marl (DE); Christoph Hiller, Dülmen (DE); Franz-Felix Kuppinger, Marl (DE); Sebastian Soboll, Recklinghausen (DE); Peter Kreis, Dortmund (DE)

(21) Appl. No.: 18/841,337

(22) PCT Filed: Feb. 15, 2023
(86) PCT No.: PCT/EP2023/053747

§ 371 Date: Aug. 23, 2024

(30) Foreign Application Priority Data
Feb. 25, 2022 (EP) 22158781.9

(51) Int. Cl. C07C 51/25; C07C 51/44; C07C 51/47

(52) U.S. Cl. CPC C07C 51/252; C07C 51/44; C07C 51/47

(57) ABSTRACT

The invention relates to the extraction of acrylic acid from aqueous flows. It is based on the object of specifying a method for extracting acrylic acid from aqueous flows, which manages with a low energy consumption and which permits longer operating times. This object is achieved in that to extract the acrylic acid, the aqueous flow containing the acrylic acid is brought into contact with a membrane, the side of which facing away from the aqueous flow is subjected to an organic solvent.

Acrylic acid is a basic chemical which is industrially produced in a large scale. It is used as a monomer for producing acrylates, among other things.

An overview of the production of acrylic acid and esters derived therefrom is offered by:

Ohara, T., Sato, T., Shimizu, N., Prescher, G., Schwind, H., Weiberg, O., Marten, K., Greim, H., Shaffer, T. D. and Nandi, P. (2021). Acrylic Acid and Derivatives. In Ullmann's Encyclopedia of Industrial Chemistry. https://doi.org/10.1002/14356007.a01_161.pub4.

One industrially significant synthetic route is to oxidize propene in a first step to form acrolein and then in a second step to form acrylic acid. The oxidation steps can be carried out in separate reactors on different catalysts or integrated in a reactor on one catalyst. In general, the reaction takes place in the gas phase. The reaction gas obtained also contains many further byproducts in addition to the desired acrylic acid. Since the byproducts very strongly impair the usability of the acrylic acid as a monomer for producing high-quality acrylates, it is a significant technical development goal of acrylic acid production to separate the acrylic acid as purely as possible from the reaction gas.

For this purpose, the reaction gas is usually contacted with a liquid absorber (generally water or an organic solvent), so that the acrylic acid is thus present together with several byproducts in a solution. The solution is then distilled in multiple steps so that concentrated acrylic acid (crude acrylic acid-AA) is obtained. This is then purified further using crystallization processes so that at the end high-purity acrylic acid (glacial acrylic acid-GAA) is then provided.

This process has proven itself in decades of industrial practice. One disadvantage in principle is that acrylic acid is extremely reactive and even tends toward self-polymerization. Great effort therefore has to be taken to avoid the reaction of the acrylic acid with itself during its processing. This is carried out, inter alia, by adding inhibitors, which suppress the reaction. However, this is not always completely successful, so that polyacrylates accumulate in the distillation column over a longer operating time. To maintain the operational reliability, the distillation of the acrylic acid has to be regularly interrupted, the columns have to be emptied and manually cleaned of polyacrylates. This is a very complex process which noticeably increases the costs for processing the acrylic acid. In addition, a very large amount of thermal energy is required for the distillation of acrylic acid, which increases the production costs and enlarges the CO₂ footprint of the acrylic acid and its derived products.

Document DE 2364679 discloses an extraction process for separating acrylic acid from an aqueous medium using a selective membrane. Direct contact of the aqueous medium with the other phase into which the acrylic acid diffuses is prevented by the selective membrane. The acrylic acid initially diffuses from the aqueous medium into the selective membrane and subsequently into the other phase. The diffusion resistance is in this case relatively high and the diffusion proceeds slowly and energy-inefficiently. To ramp up the diffusion the membrane surface area could theoretically be increased, though this would result in elevated material requirements, elevated costs and a more complex construction of the membrane apparatus.

There is therefore great interest in making possible the purification of acrylic acid in a less complex and simultaneously more energy-efficient manner.

One possibility for reducing the use of thermal energy is the use of extraction processes. In extraction processes, an aqueous absorber containing the acrylic acid is contacted with an organic extraction medium. The acrylic acid is thus enriched in the organic solvent. A separation of the organic phase from the aqueous phase is then also required and finally the distillation of the organic phase to obtain the acrylic acid. One advantage of the described extraction process over pure distillation is that less thermal energy is required. This is because the solvent generally has a lower evaporation enthalpy than water. The distillative separation of the acrylic acid from the solvent is therefore more energetically favourable than the separation of acrylic acid from water.

One method for extracting acrylic acid using organic solvents is described in EP1466885A2. A mixture made of isopropyl acetate and toluene is used as the extraction medium, a KARR column is mentioned as a contactor. In KARR columns, the organic and the aqueous phase are dispersed by rotating actuators to achieve a large contact surface at the phase boundaries. One disadvantage in principle of the KARR columns is again the demand for mechanical energy which is required to rotate the actuators. In addition, the moving parts of the KARR column are particularly susceptible to polymers: Therefore, KARR columns have comparatively tight shutdown times in acrylic acid. In addition, a KARR column furthermore requires a phase separation after completed extraction.

Membrane separation comes into consideration as a further possibility for separating acrylic acid from aqueous flows. Thus, U.S. Pat. No. 5,635,071A describes the separation of carboxylic acids from aqueous flows with the aid of a nanofiltration membrane. The method is pressure-driven, preferably at 300 psig transmembrane pressure and is carried out at a temperature of 40 to 50° C. Specifically, a mixture of acetic acid and formic acid is separated by the membrane. The membrane material is not specified precisely; the usability of a desalination membrane from Desalination Systems Inc. of California, available under the model number DS-5-DK8040, is merely noted. What type of membrane material this is specifically is not stated. The selection of the membrane material is of decisive interest for a person skilled in the art in the field of membrane technology. The content of the disclosure of U.S. Pat. No. 5,635,071A is thus incomplete. In addition, it is also unclear whether the desalination membrane is also suitable for separating acrylic acid, because this was not investigated in the specification. Finally, the temperature at which the nanofiltration is carried out is still quite high, so that an increased tendency toward polymerization is to be expected. This effect would also have to be strengthened if the nanofiltration membrane were impermeable to the inhibitor. In this case, autopolymerization of the acrylic acid will certainly be expected on the permeate side. As a result, it is doubtful whether the technical teaching disclosed in U.S. Pat. No. 5,635,071A is sufficient to implement membrane-assisted separation of acrylic acid from aqueous solutions.

With regard to this prior art, the invention is based on the object of specifying a method for extracting acrylic acid from aqueous flows which manages with low energy consumption and which permits longer operating times.

This object is achieved in that the aqueous flow containing the acrylic acid is brought into contact with a membrane, the side of which facing away from the aqueous flow is subjected to an organic solvent.

Such a method is a first subject matter of the invention.

The invention is based on the basic concept that a so-called membrane contactor may be used for extracting the acrylic acid. A membrane contactor is an apparatus which is divided by a porous membrane into two compartments: The so-called shell and the so-called lumen. The shell lies on this side of the membrane, while the lumen lies beyond the membrane. The membrane has a differing wettability for organic and aqueous media based on the material. If the two compartments are respectively subjected to the aqueous medium or the organic medium, the pores of the membrane fill with the phase for which the membrane material has a higher wettability. No mixing occurs due to the surface tension between the two media at the interface: The surface tension of the two media in relation to one another prevents breaking through. The contact surface required for the extraction between the two phases is defined via the pores of the membrane. As a result, the membrane contactor enables a liquid/liquid extraction from the aqueous phase into the organic phase at a defined contact surface without mixing the phases.

The advantage of the membrane contactor is therefore that an extraction can be carried out without having to perform a subsequent phase separation.

Various membrane apparatuses are known and differ in terms of the mass transfer mechanism and the employed membrane and can therefore be divided into the following three groups: one group utilizes diffuse mass transfer through a nonporous membrane to separate substances. This group includes gas permeation, pervaporation and reverse osmosis. A further group is based on the principle of convective mass transfer through a porous membrane, as used in ultrafiltration and microfiltration. In contrast thereto, membrane contactors represent a third group which is based on the principle of diffuse mass transfer through a porous membrane. Here, one phase wets the membrane contactor and fills the pores thereof. Therefore, both phases are in direct contact, as a result of which the substance to be separated diffuses directly from one phase into the other phase through the pores of the membrane contactor without first diffusing into the membrane. As a result, the diffusion resistance is reduced and the substance to be separated diffuses faster/a smaller contact surface for separation is required. The membrane contactor itself serves merely to stabilize the phase interface. Compared to other membrane apparatuses the membrane contactor does not utilize any specific separation property of the membrane such as selectivity or cutoff. More information about the various apparatuses and in particular about membrane contactors may be found in T. Melin, R. Rautenbach: “Membranverfahren-Grundlagen der Modul-und Anlagenauslegung” [Membrane Methods-Principles of Module and System Design], 3rd edition, Springer-Verlag Berlin Heidelberg, 2007.

Suitable membrane contactors are known, inter alia, from WO 2008088293 A1 or also from EP 3444021 B1 and are moreover commercially available.

A membrane is preferably used which contains porous polytetrafluoroethylene (PTFE) or consists thereof. The PTFE can be hydrophilised, but does not have to be. PTFE is normally hydrophobic. It is roughly unimportant whether a hydrophilised or non-hydrophilised PTFE membrane is used. It is only important in the selection of the membrane material that of two phases which are to be brought into contact on the membrane, only one wets the surface of the membrane. If the two media then also have a surface tension in relation to one another—which is the case with an organic medium on one side and an aqueous medium on the other side—this prevents the breakthrough through the membrane. Breakthrough means here that the organic phase flows through the pores into the aqueous phase. This is prevented by the higher pressure in the water and the surface tension.

In addition, it is fundamentally advantageous to select a membrane material which is wettable with the medium in which the diffusion coefficient of the component to be exchanged is higher.

However, the porosity of the membrane material is important. The separation-active material preferably has a porosity of 25% to 75%. The porosity is preferably approximately 50%. The pores are to have a pore diameter between 0.2 μm and 0.4 μm. The membrane preferably consists completely of correspondingly porous PTFE.

The membrane is preferably provided as a hollow fibre module. Hollow fibre modules comprise a bundle of a plurality of hollow fibre membranes connected in parallel. A hollow fibre membrane has the form of a pipe, on the lumen side (inside) of which one medium flows, while the other medium flows on the outside (shell). The shell sides of all hollow fibres form a common space, whereas each hollow fibre has an individual lumen. The aqueous flow is preferably located on the lumen side while the organic solvent flows on the shell side. The reverse is also possible. Organic solvent and aqueous flow preferably flow in opposite directions through the hollow fibre module (counter flow operation). The concentration gradient is thus maintained better.

One advantage of the extraction method is that it may be carried out at ambient temperature. Thermal energy is thus saved in comparison to distillation. The extraction is preferably carried out at a temperature of 20° C. to 30° C., because then generally no thermal energy is required. In addition, the tendency toward polymerization of the acrylic acid is less pronounced at such low temperatures, which increases the operational reliability, lengthens the shutdown intervals, and reduces the need for inhibitor. The method can alternatively also be carried out at a temperature of 20° C. to 50° C.

A substance which absorbs acrylic acid well, on the one hand, but does not attack the membrane, on the other hand, is used as the organic solvent. In particular toluene, n-heptane, isobutyl acetate, acetic-n-propylester, isopropyl acetate, 2-pentanone, and methylisobutyl ketone are suitable as organic solvents. Mixtures of these substances can also be used as solvents.

Toluene is preferably used as an organic solvent. Toluene meets the requirements very well and is easily available as a mass-produced chemical. A mixture with further substances is then not necessary. The fresh organic solvent therefore preferably consists of 95 wt. % to 100 wt. % toluene. The remainder can be contaminants.

It is to be made clear that the membrane used in the present method is unmoving. This means that no mechanical power is necessary to move the membrane in relation to the surroundings. There is thus an advantage over a KARR column, which comprises moving parts. In addition, an unmoving membrane is less susceptible to blockage due to undesired polymerization. The mechanical power required for the flow of the aqueous medium through the membrane is negligible, because the aqueous flow has to be pumped in a continuous process in any case.

According to one preferred refinement of the method, the organic solvent is circulated in the cycle, wherein the cycle comprises the subjection of the membrane to the organic solvent and distillative separation of the organic solvent from the acrylic acid. The closed recirculation of the toluene may be incorporated particularly well in a continuous process for acrylic acid or acrolein production. The toluene is not lost from the system, due to which the process is particularly sustainable. This process is particularly suitable for purifying the valuable material acrylic acid of toluene.

There are various decision criteria about which medium is guided on the lumen side and which on the shell side. The interface can be observed for this purpose. The hollow fibres have a higher diameter on the outside than on the inside. If the interface is thus on the outside (wetting medium is moved on the lumen side), the interface is thus somewhat higher than in reverse. Another aspect is the flow-dynamics consideration. The flow is more linear in the lumen than on the shell. For this reason, it makes sense to guide the flow to be extracted through the lumen to prevent back-mixing due to turbulence in the flow with less enriched product before the exit from the contactor. In view of these considerations, the organic solvent is preferably moved on the shell side, while the aqueous flow is moved on the lumen side.

If a hydrophobic membrane is used, it is necessary to move the aqueous flow at a higher pressure through the membrane module than the organic solvent. Specifically, it is preferred to carry out the process using a pressure gradient which prevails between the aqueous side and the organic side of the membrane, wherein the pressure on the aqueous side is between 1000 Pa and 10 000 Pa higher than the pressure on the organic side.

Such a slight overpressure on the side of the non-wetting medium prevents the wetting liquid from breaking through. With such a non-hydrophilised membrane, a higher pressure is thus always to prevail on the side of the aqueous phase than on the side of the wetting (organic) phase. When starting the process, it is therefore necessary to begin on the side of the aqueous phase. If one were to start with the wetting phase, this would flow through the pores to the other side of the membrane, because there is no counter pressure. Accordingly, it is unimportant whether the higher pressure is on the shell side or lumen side. It is only important that the pressure is higher on the side of the non-wetting liquid than on the wetting side. Moreover, the pressure gradient is not the driving force. The driving force is the concentration gradient.

One preferred refinement of the method according to the invention therefore provides a pressure gradient which prevails between the two sides of the presently non-hydrophilised membrane, wherein the pressure on the (organic) side facing away from the aqueous flow is between 1000 Pa and 10 000 Pa lower than the pressure on the (aqueous) side facing toward the aqueous flow.

If a hydrophilised membrane is used, the pressure gradient is to be reversed accordingly.

One alternative refinement of the method according to the invention therefore provides a pressure gradient which prevails between the two sides of a hydrophilised membrane, wherein the pressure on the (organic) side facing away from the aqueous flow is between 1000 Pa and 10 000 Pa higher than the pressure on the (aqueous) side facing toward the aqueous flow.

To avoid the undesired polymerization of acrylic acid during the extraction, carrying out the extraction in the presence of an inhibitor is indicated. The inhibitor has to be present on both sides of the membrane. Due to the aqueous environment on one side and the organic environment on the other, it is possible to use different inhibitors which are accordingly more stable in aqueous or organic environment, respectively. One refinement of the method accordingly provides that the aqueous flow contains a first inhibitor and that the organic solvent contains a second inhibitor, wherein the first inhibitor and the second inhibitor are the same or different.

In particular hydroquinone (1,4-dihydroxybenzene) and/or hydroquinone monomethylether (MEHQ) come into consideration as the first inhibitor in the aqueous flow, 4-hydroxy-2,2,6,6-tetramethyl piperidinyloxyl (4-HT) can be used as the second inhibitor in the organic solvent.

The extraction method described here can also be combined with classic extraction methods including phase separation, for example, if acrylic acid is to be separated in quantities for which the performance of the membrane contactors is not sufficient. In a first step, a classic extraction is then carried out using phase separation and then in a second step extraction is carried out according to the invention using membrane contactor and without phase separation. The second extraction step according to the invention would then represent a fine purification. Ultimately, the selection of the matching setup is a question of cost effectiveness.

The extraction method described here moreover also functions in the opposite direction, namely to extract acrylic acid from an organic flow into an aqueous flow. Surprisingly, the reverse path even functions without setting the wettability of the membrane in reverse. Apparently, it is unimportant on which side the membrane has which wettability; it is decisive that on one side the wettability of the membrane for water is greater than for the organic solvent.

A second subject matter of the invention is therefore methods for extracting acrylic acid from organic flows, characterized in that an organic flow containing acrylic acid is brought into contact with a membrane, the side of which facing away from the organic flow is subjected to an aqueous solvent.

In a preferred embodiment of the invention, the membrane is a membrane contactor.

According to the mode of operation of a membrane contactor, within the membrane contactor, the aqueous stream is in direct contact with the organic solvent or the organic stream is in direct contact with the aqueous solvent. Mass transfer accordingly occurs directly between the organic phase and the aqueous phase without the detour of the membrane material.

The extraction method according to the invention is preferably used in the industrial production of acrylic acid.

A further subject matter of the invention is therefore a method for producing acrylic acid, comprising a first reaction step in which propene is reacted with oxygen to form acrolein, a second reaction step in which acrolein is reacted with oxygen to form acrylic acid, an absorption step in which acrylic acid is absorbed in an aqueous flow, and an extraction step in which the acrylic acid is extracted from the aqueous flow in the way described here into an organic solvent.

Specifically, the membrane contactor can be used instead of a KARR column. Alternatively, a combination of an extraction column without energy introduction can be used in series with a membrane contactor. Moreover, the membrane contactor can be used in the wastewater flows of the acrylic acid production to reclaim residual acrylic acid with the aid of an organic solvent.

The extraction method according to the invention can also be used in the industrial production of acrolein. A further subject matter of the invention is therefore a method for producing acrolein, comprising a reaction step in which propene is reacted with oxygen to form acrolein and acrylic acid and an absorption step in which acrylic acid is absorbed in an aqueous flow and an extraction step in which the acrylic acid is extracted from the aqueous flow in the way described here into an organic solvent.

The membrane contactor can be used in particular in the wastewater flows of the acrolein production to reclaim residual acrylic acid with the aid of a solvent.

In a similar manner, the method according to the invention may be used in the production of acrylates, in particular butyl acrylate, because in such methods aqueous flows containing acrylic acid also occur. The acrylic acid can be reclaimed using the extraction method according to the invention. The invention is to be explained in more detail on the basis of an exemplary embodiment. In the figures:

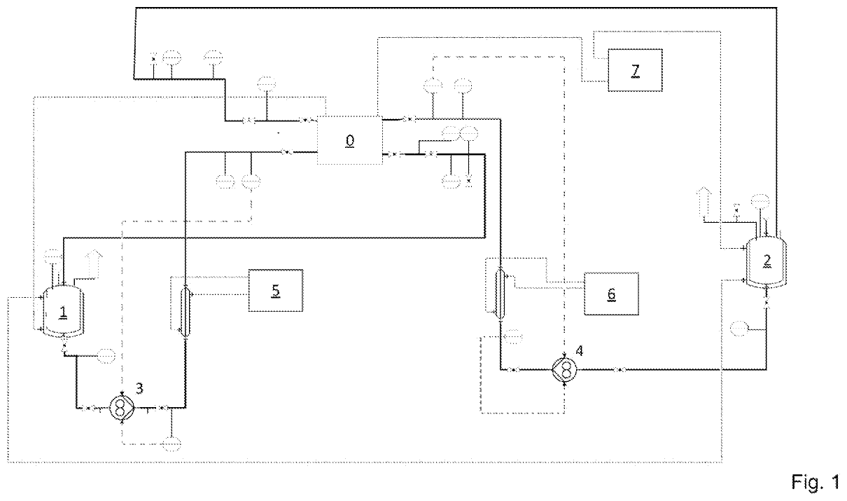

FIG. 1: shows a simplified flow chart of the experimental setup;

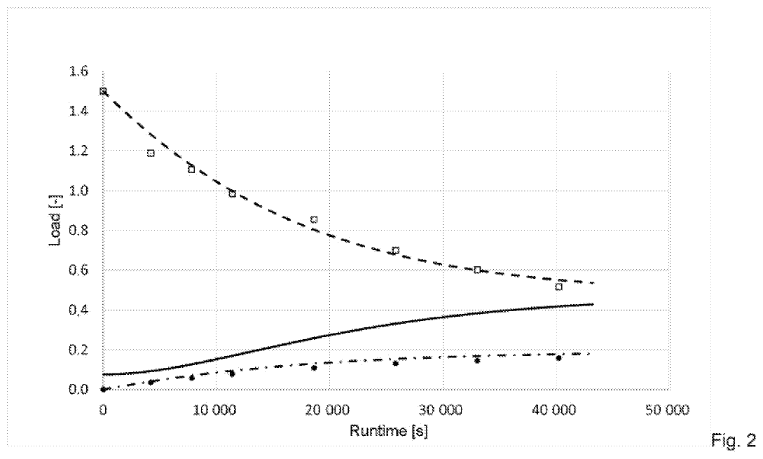

FIG. 2: shows the load progression of the acrylic acid in aqueous and organic phase;

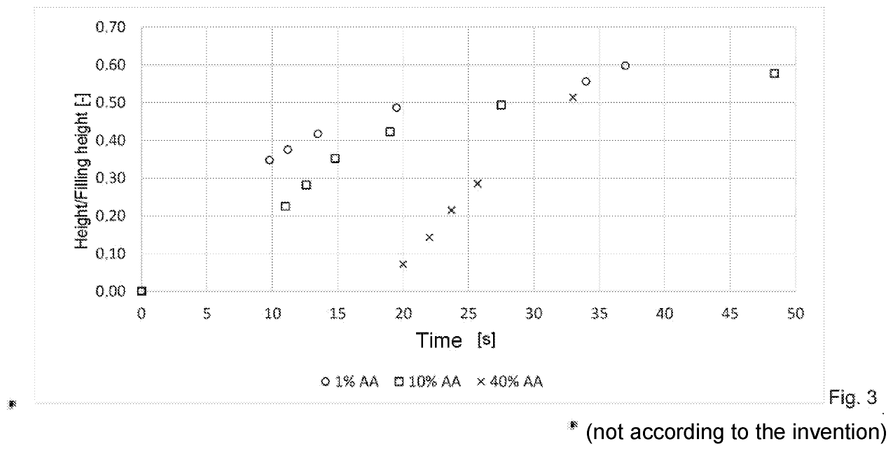

FIG. 3: shows the level of the sedimentation line against time after dispersion in shaking experiments (not according to the invention).

In the application considered here, acrylic acid is to be extracted from an aqueous flow.

The extraction of acrylic acid from water with the aid of toluene at a PTFE membrane was observed as an example. Specifically, a membrane contactor in the form of a hollow fibre module was used, acquired from Memo3 GmbH, CH-4313 Mohlin. The application in the other direction (extraction of acrylic acid from a PTFE-wetting flow with the aid of water) is also conceivable. Using a hydrophilised PTFE membrane is also conceivable. The extraction of 60% acrylic acid in water using toluene has functioned in the experiments. In these experiments, the two solutions were moved at room temperature in cyclic operation from their storage again and again over the contactor and subsequently collected again in the same storage.

A simplified method flow chart of the experimental setup is shown in FIG. 1.

The central element is the studied membrane contactor 0. The membrane contactor 0 has no moving parts. In a first container 1, water having the respective proportion of acrylic acid is provided. In a second container 2, toluene is located as an organic solvent. An aqueous flow containing acrylic acid is conveyed from the first container 1 into the lumen of the membrane contactor 0 using a first pump 3. The organic solvent (toluene) is conveyed using a second pump 4 from the second container 2 into the shell of the membrane contactor 0, so that an organic flow results.

With the aid of a first thermostat 5 having connected heat exchanger, the temperature of the aqueous flow is set in the lumen of the membrane contactor 0. With the aid of a second thermostat 6 having connected heat exchanger, the temperature of the organic solvent is set in the shell of the membrane contactor 0. A third thermostat 7 sets the temperature of the membrane contactor 7 and the two containers 1, 2. The measurement and regulation lines of the temperature regulation are shown by dotted lines in FIG. 1.

In addition to the temperature regulation with the aid of the three thermostats 5, 6, 7, the experimental setup also has a flow regulation which regulates the pressures and volume flows of the aqueous flow and the organic flow. This takes place via the respective pumps 3, 4 and numerous valves which are shown in FIG. 1 but are not numbered. The associated regulators and sensors are also shown but are not individually numbered. The measurement and regulation lines of the flow regulation are shown by dashed lines in FIG. 1.

The lines through which the aqueous and organic flows flow are shown by solid lines in FIG. 1. The aqueous flow containing acrylic acid and the organic solvent flow in counter flow through the membrane contactor 0 and are recycled in their respective containers 1, 2.

The extraction of the acrylic acid from the aqueous flow into the organic solvent takes place in the membrane contactor 0. The acrylic acid travels from the lumen side to the shell side of the membrane. Acrylic acid is depleted in the water and enriched in the toluene.

In the first container 1, approximately 1 kg 60% acrylic acid in water, stabilized with 500 ppm 4-hydroxy-2,2,6,6-tetramethyl piperidinyloxyl (4-HT), was provided and guided in the cycle via the contactor 0. The aqueous phase is moved in the contactor embodied as a hollow fibre module on the lumen side. On the shell side, toluene is moved in the cycle, also stabilized with 500 ppm 4-HT. The toluene is provided in the second container 2 at approximately 2 L and moved in the cycle via the contactor. The aqueous phase and the organic phase are contacted with one another in counter flow in the membrane contactor 0. The experiment takes place at 25° C. On the lumen side, a pressure of approximately 65 mbar (6500 Pa above the pressure on the shell side) is set to prevent the toluene from breaking through. The two containers 1 and 2 were sampled regularly to determine the acrylic acid proportion in the two solutions.

In the diagram shown in FIG. 2, the experimentally determined loads of the two phases are shown. In the diagram, the mass load is recorded over the runtime of the experiment. The square points (a) relate to the aqueous phase, the round points (●) relate to the organic phase. Interpolated by computer, the load for the aqueous phase is shown as the dashed line. Interpolated by computer, the load for the organic phase is shown as a dot-dash line. The calculated equilibrium line for the aqueous phase is shown as a solid line.

The experiment has shown that a depletion of the acrylic acid from the water using toluene as the extraction agent has taken place via the membrane contactor within 11 hours nearly to the first equilibrium point. In the 11 hours, no separating performance collapse, for example, due to occurring polymerization and possibly linked collapse of the porosity of the membrane, could be observed. No swelling of the membrane could be visually determined either. No polymerization occurred. This can be explained in that the experiment took place at room temperature and the inhibitors in both solutions prevent polymerization.

The feasibility of the extraction of acrylic acid from water with the aid of toluene via a membrane contactor with inhibition by means of 4-HT has been shown by this experiment. It was possible to determine a mass transfer coefficient k at a level of 2.4*10⁻⁶ m/s, the theoretical concentration progressions of which of acrylic acid in water and toluene correspond with only minor deviations to the experimental data. The theoretical curves determined with the aid of the mass transfer coefficient are shown in FIG. 2 in the dashed lines. The determined equilibrium load in the aqueous phase is shown in the solid line for each point in time.

The most important experimental data are shown in Tables 1a to 1d. Table 1a shows the formulations used in the experiment, Table 1b shows the flow rates in the experimental procedure, Table 1c lists the general method guidelines. The experimental results are compiled in Table 1d.

TABLE 1a
Formulations in the experimental procedure

| | Toluene | AA | Water | 4HT |
|---|---|---|---|---|
| Aqueous | | 60% | 40% | 500 ppm |
| phase | | | | |
| Organic | 100% | | | 500 ppm |

TABLE 1b
Flow rates in the experimental procedure

| | | | | | Flow rate |
|---|---|---|---|---|---|
| | | | | | Target |
| | | | | | kg/h |
| Weight of sample | 998 | g | (target = 1 kg) | Lumen | 5 |
| aqueous phase: | | | | | |
| Weight of sample | 2135 | g | (target = 2.1 kg) | Shell | 5 |
| organic phase | | | | | |

TABLE 1c
Method guidelines

| | | |
|---|---|---|
| Experimental | 25° | C. |
| temperature: | | |
| Experimental duration: | 24 | h |

| | |
|---|---|
| Sample interval | every 2 hours |
| Sample quantity | 10 mL for aqueous phase |
| | 10 mL for organic phase |
| Comments: | 1 L stopper solution (water with |
| | 500 ppm 4-HT) provided |

TABLE 1d
Experimental results

| | PIR301 | PIR302 | | | | | FIR 202 | FIR 204 |
|---|---|---|---|---|---|---|---|---|
| Experiment | Lumen | Lumen | PIR303 | | Level | Level | Density | Density |
| time | in | out | Shell in | Differential | Lumen | Shell | of lumen | of shell |
| hh:mm | mbar | mbar | mbar | pressure | cm | cm | g/mL | g/mL |
| | 1.070 | 1.01 | 1.024 | 0.046 | 5 | 15 | 1.043 | 0.861 |
| 1 | 1.070 | 1.01 | 1.018 | 0.052 | 5.3 | 14.6 | 1.042 | 0.862 |
| 2 | 1.055 | 1.01 | 1.016 | 0.039 | 6.2 | 14 | 1.041 | 0.863 |
| 3 | 1.064 | 1.01 | 1.016 | 0.048 | 7.2 | 13.6 | 1.039 | 0.864 |
| 4 | 1.061 | 1.01 | 1.018 | 0.043 | 7.9 | 13.5 | 1.039 | 0.868 |
| 5 | 1.060 | 1.01 | 1.018 | 0.042 | 8.4 | 13 | 1.039 | 0.870 |
| 6 | 1.064 | 1.01 | 1.019 | 0.045 | 9 | 12.8 | 1.039 | 0.872 |
| 7 | 1.064 | 1.01 | 1.019 | 0.045 | 9.3 | 12.5 | 1.038 | 0.873 |
| 8 | 1.060 | 1.01 | 1.019 | 0.041 | 10 | 12.5 | 1.037 | 0.874 |
| 9 | 1.062 | 1.01 | 1.0172 | 0.045 | 10.5 | 12.5 | 1.03544 | 0.875 |
| 10 | 1.062 | 1.01 | 1.0167 | 0.045 | 11 | 12.5 | 1.0344 | 0.876 |
| 11 | 1.060 | 1.01 | 1.0169 | 0.043 | 11.5 | 12.5 | 1.03336 | 0.877 |

The extraction was carried out without membrane in shaking experiments as a comparison. These were carried out using various acrylic acid concentrations in water and toluene as the extracting agent in the ratio 2:1 (water:toluene). With equal introduction of energy, the two-phase solutions made of acrylic acid, water, and toluene (stabilized with 4-HT) were dispersed and the settling behaviour was subsequently observed.

In the diagram shown in FIG. 3, the level at which the phase boundary was located is plotted against the time after dispersion. The height is related to the total filling height and is therefore dimensionless. The time is indicated in seconds. The round points (o) relate to the measurement points with 1% acrylic acid (AA), the square points (□) relate to the measurement points with 10% acrylic acid (AA), the cross points (x) relate to the measurement points with 40% acrylic acid (AA). The respective points form a sedimentation line (not shown).

The sedimentation speed multiplies upon reduction of the acrylic acid concentration used in water from 40% (0.6 mm/s) to 1% (2.4 mm/s). The associated data are shown in Table 2.

Normally, a large amount of energy has to be applied in a system made up of water and toluene to produce drops, because the surface tension is so high. Therefore, KARR columns were also used in the prior art for this extraction task—with the disadvantages described at the outset. It was shown by the present shaking experiments that due to the presence of “a large amount” of acrylic acid the surface tension is apparently reduced and drops can be produced with less introduction of energy. Accordingly, the required energy introduction for the dispersion with the 1% acrylic acid in water with toluene is significantly higher than in the case of the 40% acrylic acid in water with toluene.

A combination of column and membrane contactor, depending on the energy introduction required, can therefore be reasonable. The optimum results from a cost effectiveness calculation.

TABLE 2
Sedimentation speed as a function of the concentration
used of the acrylic acid in aqueous phase in a
phase ratio of aqueous phase to toluene.

| | Density difference aqueous | Sediment speed |
|---|---|---|
| | phase - organic phase | [mm/s] |
| 1% acrylic acid | 133 | 2.4 |
| 10% acrylic acid | 129 | 1.6 |
| 40% acrylic acid | 117 | 0.6 |

### REFERENCE SIGNS

0 membrane contactor
1 first container (lumen-water/acrylic acid)
2 second container (shell-toluene)
3 first pump
4 second pump
5 first thermostat with heat exchanger
6 second thermostat with heat exchanger
7 third thermostat

1. Method for extracting acrylic acid from aqueous flows, in which an aqueous flow containing acrylic acid is brought into contact with a membrane, the side of which facing away from the aqueous flow is subjected to an organic solvent, wherein the membrane is a membrane contactor.

2. Method according to claim 1, wherein the membrane contains porous polytetrafluoroethylene or in that the membrane consists of porous polytetrafluoroethylene.

3. Method according to claim 1, wherein the membrane is provided in the form of a hollow fibre module, which is operated in counter flow.

4. Method according to claim 1, wherein it is carried out at a temperature of 20° C. to 30° C. or of 20° C. to 50° C.

5. Method according to claim 1, wherein the organic solvent contains at least one substance selected from the group consisting of toluene, n-heptane, isobutyl acetate, acetic-n-propylester, isopropyl acetate, 2-pentanone, and methylisobutyl ketone.

6. Method according to claim 5, wherein toluene is used as an organic solvent.

7. Method according to claim 1, wherein the membrane is unmoving.

8. Method according to claim 1, wherein the organic solvent is circulated in the cycle, wherein the cycle comprises the application of the organic solvent to the membrane and distillative separation of the organic solvent from the acrylic acid.

9. Method according to claim 1, wherein the organic solvent is moved on the shell side, while the aqueous flow is moved on the lumen side.

10. Method according to claim 1, carried out using a hydrophobic membrane, characterized by a pressure gradient which prevails between the one and the other side of the membrane, wherein the pressure on the side facing away from the aqueous flow is between 1000 Pa and 10 000 Pa lower than the pressure on the side facing toward the aqueous flow.

11. Method according to claim 1, carried out using a hydrophilized membrane, characterized by a pressure gradient which prevails between the one and the other side of the membrane, wherein the pressure on the side facing away from the aqueous flow is between 1000 Pa and 10 000 Pa higher than the pressure on the side facing toward the aqueous flow.

12. Method according to claim 1, wherein the aqueous flow contains a first inhibitor and in that the organic solvent contains a second inhibitor, wherein the first inhibitor and the second inhibitor are the same or different.

13. Method according to claim 1, wherein there is at least one direct contact between the aqueous flow and the organic solvent inside the membrane contactor.

14. Method for extracting acrylic acid from organic flows, in which an organic flow containing acrylic acid is brought into contact with a membrane, the side of which facing away from the organic flow is subjected to an aqueous solvent, wherein the membrane is a membrane contactor.

15. Method according to claim 14, wherein there is at least one direct contact between the organic flow and the aqueous solvent inside the membrane contactor.

16. Method for producing acrylic acid, comprising a first reaction step in which propene is reacted with oxygen to form acrolein, a second reaction step in which acrolein is reacted with oxygen to form acrylic acid, and an absorption step in which acrylic acid is absorbed in an aqueous flow, characterized by an extraction step in which acrylic acid is at least partially extracted from the aqueous flow by bringing the aqueous flow into contact with a membrane, the side of which facing away from the aqueous flow is subjected to an organic solvent, wherein the membrane is a membrane contactor.

17. Method for producing acrolein, comprising a reaction step in which propene is reacted with oxygen to form acrolein and acrylic acid and furthermore comprising an absorption step in which acrylic acid is absorbed in an aqueous flow, characterized by an extraction step in which acrylic acid is at least partially extracted from the aqueous flow by bringing the aqueous flow into contact with a membrane, the side of which facing away from the aqueous flow is subjected to an organic solvent, wherein the membrane is a membrane contactor.
